(19) United States
(12) Patent Application Publication
(10) Pub. No.: US 2006/0074057 A1
(43) Pub. Date: Apr. 6, 2006

(54) Use of chenodeoxycholic acid for reducing adipose tissue

(76) Inventor: Eric Marchewitz, Novi, MI (US)

Correspondence Address: Miller, Canfield, Paddock and Stone P.L.C.;c/o Robert Kelley Roth, Suite 2500, 150 West Jefferson Ave, Detroit, MI, 48226, US

(21) Appl. No.: 11/242,747

(22) Filed: Oct. 4, 2005

Related U.S. Application Data
(60) Provisional application No. 60/616,145, filed on Oct. 4, 2004.

(51) Int. Cl. A61K 31/56

(52) U.S. Cl. 514/169

(57) ABSTRACT

Use of chenodeoxycholic acid is disclosed to reduce adipose tissue and thereby reduce weight in mammals. The chenodeoxycholic acid can be administered orally through the use of a tablet, pill, capsule or liquid suspension.

### RELATED APPLICATION

This application claims priority benefit of U.S. provisional patent application No. 60/616,145 filed on Oct. 4, 2004.

### FIELD OF THE INVENTION

This invention relates to a new method of use of chenodeoxycholic acid, and more particularly, using chenodeoxycholic acid for reducing adipose tissue and body weight in humans.

### BACKGROUND OF THE INVENTION

Maintaining relatively low adipose tissue as a percentage of total body weight is important for maintaining health in humans. The benefits are well known and include reduced cholesterol levels, lowered blood pressure, increased insulin sensitivity, increased glucose disposal and proper regulation of glucose levels. There are numerous disease states associated with being overweight and with high levels of adiposity. These include cancer, high blood pressure, high cholesterol, chronic renal failure, congestive heart failure and type II diabetes. There are several known methods for reducing adipose tissue and thereby reducing body weight through the use of pharmaceuticals and through dietary supplements. Appetite suppressants and stimulants include Meridia and Amphetamines, and dietary supplements include Hoodia and Epehedra. Unfortunately, appetite suppressants alone do not have a high rate of success and are often accompanied by a rebound weight gain when their use is stopped. Stimulants are also known to have some serious side effects, including strain on the cardiovascular system and as such, their use is limited.

Another method for weight reduction involves the use of supplements that regulate cortisol levels in the body. Cortisol is a stress hormone that is produced in the body and released in times of stress. Its primary purpose is to cause a release of sugars into the blood stream and provide increased energy for the body to deal with stressful situations. Cortisol also has the effect of increasing appetite for certain foods such as carbohydrates and sugars to provide sources of energy for the future. In situations where the stress is longer term the human body may produce too much cortisol. This can result in increased hunger and corresponding food intake which can lead to the buildup of undesirable fat tissues, particularly in the abdominal area. Thus, the presence of elevated levels of cortisol for long periods of time can result in undesirable weight gain. Many existing products claim to reduce cortisol levels to assist in weight reduction. Typically these products work by inhibiting cortisol production. However, the body may ultimately compensate for this reduction by increasing the production of cortisol if stress remains present. This can result in the return of unwanted fat tissues.

Chenodeoxycholic acid is a naturally occurring bile acid heretofore has not been known as a treatment for reduction of adipose tissue. However, this compound has been found useful in other areas. For example, U.S. Pat. No. 5,310,560 to Widauer discloses a method for using chenodeoxycholic acid for the treatment acute or chronic inflammatory illnesses of the respiratory organs. U.S. Pat. No. 4,681,876 to Marples et al. discloses its use as an anti-fungal antibiotic.

### SUMMARY OF THE INVENTION

In accordance with a first aspect, a method for decreasing adipose tissue in humans comprises administering chenodeoxycholic acid. Preferably chenodeoxycholic acid is administered orally in a dosage between 25 and 500 mg daily.

From the foregoing disclosure and the following more detailed description of various preferred embodiments it will be apparent to those skilled in the art that the present invention provides a significant advance in the methods for treating obesity. Particularly significant in this regard is the potential the invention affords for providing an improved method of reducing adipose tissue. Additional features and advantages of various preferred embodiments will be better understood in view of the detailed description provided below.

### DETAILED DESCRIPTION OF CERTAIN PREFERRED EMBODIMENTS

It will be apparent to those skilled in the art, that is, to those who have knowledge or experience in this area of technology that many variations are possible for the method of reducing adipose tissue disclosed here. The following detailed discussion of various alternative and preferred features and embodiments will illustrate the general principles of the invention with reference to improved method of reducing adipose tissue through the use of orally available dietary supplements. Other embodiments suitable for other applications will be apparent to those skilled in the art given the benefit of this disclosure.

The present invention provides a method of administering the naturally occurring bile acid chenodeoxycholic acid (3-alpha,7-alpha-dihydroxy-5-beta-cholanic acid, C₂₄H₄₀O₄, or “CDCA”), or any esters, ethers, or salts thereof, for reducing adipose tissue in mammals and thereby reducing total body weight. CDCA's structure is reproduced below.

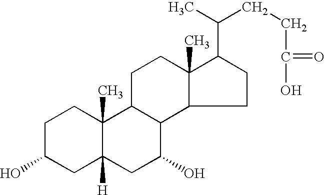

Chenodeoxycholic acid is produced naturally in human and animal livers and gall bladders. Chenodeoxycholic acid has been shown to be a selective inhibitor of 11-beta-hydroxysteroid dehydrogenase type 1 (11BHSD1). Several studies have been performed demonstrating the role of 11BHSD1 in obesity and the metabolic syndrome. 11BHSD1 is at least partially responsible for the activation of cortisol from inactive metabolites in the liver, adipose tissue and skeletal muscle. Increased cortisol levels result in increased adipose tissue deposition, breakdown of skeletal muscle tissue and disrupted glucose handling. The inhibition of 11BHSD1 with chenodeoxycholic acid advantageously results in a reduction of cortisol production principally in specific tissues rather than on a systemic basis. This can result in reduced adipose tissue in the areas of high concern, decreased skeletal muscle breakdown and better glucose handling.

Administration of chenodeoxycholic acid for reduction of adipose tissue may preferably be done orally, but it will be readily apparent to those skilled in the art that other routes of administration can be used, such as transdermally. An effective daily dosage of chenodeoxycholic acid is about 25 to 500 mg, with 100-200 mg. being most preferred. Most preferably CDCA is provided as a soft gelatin capsule or oral liquid in two to three divided doses per day. The method of reduction of adipose tissue disclosed herein is a significant improvement over existing methods, as it has better efficacy than appetite suppressants and cortisol blockers, and does not have the negative side effects associated with the use of stimulants.

For administration the active ingredient chenodeoxycholic acid thereof can be mixed with liquid carriers. It may also be delivered with a solid or semisolid carrier. CDCA may also be administered transdermally using a liquid carrier. CDCA may be delivered as in the form of an ester, ether or salt of chenodeoxycholic acid.

The amount of the active ingredient to be administered depends on various factors such as the age and weight of the user. Effective daily doses of chenodeoxycholic acid may range from 25 to 500 mg, more particularly 50 to 200 mg per day, and most preferably 100 to 200 mg per day. The dosage might be provided as a soft gelatin capsule or oral liquid. CDCA may be administered in two to three divided doses per day. Chenodeoxycholic acid may also be mixed with other weight reducing pharmaceuticals or dietary supplements such as caffeine, synephrine, or ephedrine if desired.

### EXAMPLE 1

5 kg chenodeoxycholic acid were mixed with methylcellulose, highly dispersed silicon dioxide and magnesium stearate, and placed into 100,000 hard-gelatin capsules each with a content of 50 mg chenodeoxycholic acid.

### EXAMPLE 2

5 kg chenodeoxycholic acid were mixed with 10 kg caffeine, methylcellulose, highly dispersed silicon dioxide and magnesium stearate, and placed into 100,000 hard-gelatin capsules each with a content of 50 mg chenodeoxycholic acid and 100 mg caffeine.

From the foregoing disclosure and detailed description of certain preferred embodiments, it will be apparent that various modifications, additions and other alternative embodiments are possible without departing from the true scope and spirit of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A method for decreasing adipose tissue in humans comprising administering chenodeoxycholic acid in a dosage between 25 and 500 mg daily.

2. The method of claim 1 wherein the dosage comprises between 25 and 200 mg daily.

3. The method of claim 1 wherein the dosage comprises 25 to 100 mg twice a day.

4. The method of claim 1 wherein the dosage comprises 25 to 100 mg three times a day.

5. The method of claim 1 wherein the dosage comprises 25 to 150 mg twice a day.

6. The method of claim 1 wherein the dosage comprises 25 to 150 mg three times a day.

7. The method of claim 1 wherein the chenodeoxycholic acid is administered orally.

8. The method of claim 7 wherein the chenodeoxycholic acid is administered in a gel capsule.

9. The method of claim 7 wherein the chenodeoxycholic acid is administered in a liquid suspension.

10. The method of claim 1 wherein the chenodeoxycholic acid is administered transdermally.

11. The method of claim 1 wherein chenodeoxycholic acid is administered in combination with one or more of caffeine, synephrine, or ephedrine

12. The method of claim 1 wherein the chenodeoxycholic acid is administered as one of an ester, ether or salt of chenodeoxycholic acid.

13. A method for decreasing adipose tissue in humans comprising orally administering chenodeoxycholic acid.
